(19) United States
(12) Patent Application Publication
(10) Pub. No.: US 2025/0276947 A1
(43) Pub. Date: Sep. 4, 2025

(54) FLUORINATED ALKOXYVINYL ETHERS AND METHODS FOR PREPARING FLUORINATED ALKOXYVINYL ETHERS

(71) Applicant: THE CHEMOURS COMPANY FC, LLC, Wilmington, DE (US)

(72) Inventors: VIACHESLAV A. PETROV, Hockessin, DE (US); Mario Joseph Nappa, Leesburg, FL (US); Robert Daniel Lousenberg, Wilmington, DE (US); Jonathan P. Stehman, Wilmington, DE (US)

(73) Assignee: THE CHEMOURS COMPANY FC, LLC, Wilmington, DE (US)

(21) Appl. No.: 19/214,177

(22) Filed: May 21, 2025

Related U.S. Application Data
(62) Division of application No. 18/196,761, filed on May 12, 2023, now Pat. No. 12,331,011.
Application No. 18/196,761 is a division of application No. 17/285,134, filed on Apr. 14, 2021, now Pat. No. 11,661,389.
(60) Provisional application No. 62/745,733, filed on Oct. 15, 2018.

(51) Int. Cl. C07C 43/16; C07C 41/09; C07C 43/17; C09K 5/04; C11D 7/28; C11D 7/50; H01B 3/44

(52) U.S. Cl. CPC C07C 43/16; C07C 41/09; C07C 43/17; C09K 5/048; C11D 7/28; C11D 7/5022; H01B 3/441

(57) ABSTRACT

An alkoxyvinyl ether is disclosed having the chemical structure R_{f}C(OR)═CHR_{f}′, wherein R_{f} is an at least partially fluorinated functional group having at least one carbon atom, R_{f}′ is an at least partially fluorinated functional group having at least two carbon atoms, and R is a functional group. A method for preparing an alkoxyvinyl ether is disclosed, comprising R_{f}CFHCFHR_{f}′+KOH/ROH→R_{f}C(OR)═CHR_{f}′, wherein R_{f} is a perfluoro functional group, R_{f}′ is a perfluoro functional group, and R is an alkyl functional group. Another method for preparing an alkoxyvinyl ether is disclosed, comprising R_{f}CF═CHR_{f}′+KOH/ROH→R_{f}C(OR)═CHR_{f}′, wherein R_{f} is a perfluoro functional group, R_{f}′ is a perfluoro functional group, and R is an alkyl functional group.

This application is a divisional application of U.S. application Ser. No. 18/196,761 filed May 12, 2023, allowed, which is a divisional U.S. application Ser. No. 17/285,134 filed Apr. 14, 2021, which is a U.S. national stage filing under 35 U.S.C. 371 of International Application No. PCT/US2019/055506 filed Oct. 10, 2019, and claims priority of U.S. Provisional Application No. 62/745,733 filed Oct. 15, 2018, the disclosures of which are incorporated herein by reference in its entirety.

### FIELD OF THE INVENTION

The present invention is directed to alkoxyvinyl ethers and methods for preparing alkoxyvinyl ethers. More particularly, the present invention is directed to fluorinated alkoxyvinyl ethers and methods for preparing fluorinated alkoxyvinyl ethers.

### BACKGROUND OF THE INVENTION

Certain fluorinated materials of general structure R_{f}C(OCH₃)═CFR_{f}′ are known, and such materials may be prepared by the reaction of alcohols with a variety of perfluorinated olefins, such as F-pentene-2 (known as a commercial intermediate) or F-heptenes (known as waste stream components form the production of other materials). By way of example, the product of the reaction of F-heptenes with methanol (HFX-110) has been proposed for use as a solvent with low toxicity and low global warming potential.

However, to date there has been no disclosure in the scientific literature of fluorinated chemical species having the general structure of R_{f}C(OR)═CHR_{f}′, wherein R_{f}′ has at least two carbon atoms. Nor has there been any disclosure to date of a basic alcohol reaction pathway to prepare fluorinated chemical species having the general structure of R_{f}C(OR)═CHR_{f}′, wherein R is an alkyl functional group.

### BRIEF DESCRIPTION OF THE INVENTION

In an exemplary embodiment, an alkoxyvinyl ether has the chemical structure R_{f}C(OR)═CHR_{f}′, wherein R_{f} is an at least partially fluorinated functional group having at least one carbon atom, R_{f}′ is an at least partially fluorinated functional group having at least two carbon atoms, and R is a functional group.

In another exemplary embodiment, a method for preparing an alkoxyvinyl ether includes R_{f}CFHCFHR_{f}′+KOH/ROH→R_{f}C(OR)═CHR_{f}′, wherein R_{f} is a perfluoro functional group, R_{f}′ is a perfluoro functional group, and R is an alkyl functional group.

In another exemplary embodiment, a method for preparing an alkoxyvinyl ether includes R_{f}CF═CHR_{f}′+KOH/ROH→R_{f}C(OR)═CHR_{f}′, wherein R_{f} is a perfluoro functional group, R_{f}′ is a perfluoro functional group, and R is an alkyl functional group.

Another embodiment of the invention relates to any combination of the foregoing alkoxyvinyl ethers wherein:
R_{f} is a perfluoro functional group having at least one carbon atom;
R_{f}′ is a perfluoro functional group having at least two carbon atoms; and
R is a functional group having at least one carbon atom.

Another embodiment of the invention relates to any combination of the foregoing alkoxyvinyl ethers, wherein:
R_{f} is a perfluoroalkyl functional group;
R_{f}′ is a perfluoroalkyl functional group having at least two carbon atoms; and
R is an alkyl functional group.

Another embodiment of the invention relates to any combination of the foregoing alkoxyvinyl ethers, wherein:
R_{f} is a C₁₋₁₂ perfluoroalkyl functional group;
R_{f}′ is a C₂₋₁₂ perfluoroalkyl functional group; and
R is a C₁₋₁₂ alkyl functional group.

Another embodiment of the invention relates to any combination of the foregoing alkoxyvinyl ethers, wherein:
R_{f} is selected from the group consisting of CF₃, C₂F₅, n-C₃F₇, iso-C₃F₇, n-C₄F₉, sec-C₄F₉, iso-C₄F₉, and tert-C₄F₉;
R_{f}′ is selected from the group consisting of C₂F₅, n-C₃F₇, iso-C₃F₇, n-C₄F₉, sec-C₄F₉, iso-C₄F₉, and tert-C₄F₉; and
R is selected from the group consisting of CH₃, C₂H₅, n-C₃H₇, iso-C₃H₇, n-C₄H₉, sec-C₄H₉, iso-C₄H₉, and tert-C₄H₉.

Another embodiment of the invention relates to any combination of the foregoing alkoxyvinyl ethers, wherein:
R_{f} is a partially fluorinated functional group having at least one carbon atom;
R_{f}′ is a partially fluorinated functional group having at least two carbon atoms; and
R is a functional group having at least one carbon atom.

Another embodiment of the invention relates to any combination of the foregoing alkoxyvinyl ethers, wherein:
R_{f} is a perfluoro functional group having at least one carbon atom;
R_{f}′ is a partially fluorinated functional group having at least two carbon atoms; and
R is a functional group having at least one carbon atom.

Another embodiment of the invention relates to any combination of the foregoing alkoxyvinyl ethers, wherein:
R_{f} is a partially fluorinated functional group having at least one carbon atom;
R_{f}′ is a perfluoro functional group having at least two carbon atoms; and
R is a functional group having at least one carbon atom.

Another embodiment of the invention relates to any combination of the foregoing embodiments, wherein:
R_{f} is selected from the group consisting of CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H;
R_{f}′ is selected from the group consisting of C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H; and
R is selected from the group consisting of CH₃, C₂H₅, n-C₃H₇, iso-C₃H₇, n-C₄H₉, sec-C₄H₉, iso-C₄H₉, and tert-C₄H₉.

Another embodiment of the invention relates to any combination of the foregoing embodiments wherein R_{f} and R_{f}′ are the same.

Another embodiment of the invention relates to any combination of the foregoing embodiments, wherein R_{f} and R_{f}′ are distinct.

Another embodiment of the invention relates to any combination of the foregoing embodiments, wherein the alkoxyvinyl ether is an E-isomer.

Another embodiment of the invention relates to any combination of the foregoing embodiments, wherein the alkoxyvinyl ether is a Z-isomer.

Another embodiment of the invention relates to any combination of the foregoing embodiments wherein the alkoxyvinyl ether is a mixture of E- and Z-isomers.

Another embodiment of the invention relates to any combination of the foregoing embodiments, wherein the chemical structure is CF₃C(OCH₃)═CHC₂F₅.

Another embodiment of the invention relates to any combination of the foregoing embodiments, wherein the chemical structure is CF₃C(OC₂H₅)═CHC₂F₅.

Another embodiment of the invention relates to any combination of the foregoing embodiments, wherein the chemical structure is CF₃C(OC₃H₇)═CHC₂F₅.

Another embodiment of the invention relates to any combination of the foregoing embodiments, wherein the chemical structure is C₂F₅C(OCH₃)═CHC₃F₇.

One embodiment of the invention relates to a method for preparing an alkoxyvinyl ether, comprising:

R_{f}CFHCFHR_{f}′+Base/ROH→R_{f}C(OR)═CHR_{f}′
wherein:
   R_{f} is a perfluoro functional group;
   R_{f}′ is a perfluoro functional group; and
   R is an alkyl functional group.

Another embodiment of the invention relates to a method for preparing an alkoxyvinyl ether, comprising:

R_{f}CF═CHR_{f}′+Base/ROH→R_{f}C(OR)═CHR_{f}′
wherein:
   R_{f} is a perfluoro functional group;
   R_{f}′ is a perfluoro functional group; and
   R is an alkyl functional group.

One embodiment of the invention relates to a solvent comprising any combination of the foregoing alkoxyvinyl ethers and at least one member selected from the group consisting of alcohols, ethers, tetrahydrofuran (THF), amides, or aromatic solvents.

Another embodiment of the invention relates to an alkoxyvinyl composition comprising:
any combination of the foregoing alkoxyvinyl ethers; at least one solvent including at least one of alcohols, ethers, tetrahydrofuran (THF), amides, or aromatic solvents; and
at least one base including at least one of KOH, NaOH, LiOH, Ca(OH)₂, tertiary amines, or alkoxides of alkali metals ROM (M=Li, Na, K, Cs).

Another embodiment of the invention relates to a process for transferring heat, comprising:
providing an article;
contacting the article with a heat transfer media;
wherein the heat transfer media comprises any combination of the foregoing alkoxyvinyl ethers.

Another embodiment of the invention relates to the foregoing processes, wherein the boiling point of the alkoxyvinyl ether is between about 72 degrees Celsius and about 81 degrees Celsius.

Another embodiment of the invention relates to a process for treating a surface, comprising:
providing a surface;
contacting the surface with a treatment composition;
wherein the surface includes a treatable material deposited thereon; and
wherein the treatment composition comprises any of the foregoing combinations of alkoxyvinyl ethers.

Another embodiment of the invention relates to the foregoing processes, wherein the treatment composition substantially dissolves the treatable material.

Another embodiment of the invention relates to a process for forming a composition comprising:
providing a solute;
contacting the solute with a solvent;
wherein the solvent comprises any combination of the foregoing alkoxyvinyl ethers.

Another embodiment of the invention relates to a process for providing electrical insulation, comprising:
providing a first charged surface;
providing a second charged surface;
contacting the first charged surface and the second charged surface with a dielectric composition;
wherein the dielectric composition comprises any combination of the foregoing alkoxyvinyl ethers.

Another embodiment of the invention relates to the foregoing processes, wherein the dielectric composition forms a continuous pathway between the first charged surface and the second charged surface.

The various embodiments of the invention can be used alone or in combinations with each other. Other features and advantages of the present invention will be apparent from the following more detailed description of the preferred embodiment, taken in conjunction with the accompanying drawings, which illustrate, by way of example, the principles of the invention.

### DETAILED DESCRIPTION OF THE INVENTION

Provided are exemplary fluorinated alkoxyvinyl ethers and methods for preparing fluorinated alkoxyvinyl ethers. Embodiments of the present disclosure, in comparison to compositions of matter and methods not utilizing one or more features disclosed herein, include advantageous solvating properties, advantageous boiling point ranges, advantageous degreasing properties, advantageous heat transfer properties, decreased flammability, decreased atmospheric life time, increased polarity, or combinations thereof.

In some embodiments, the alkoxyvinyl ethers may find useful application as a cleaning agent or degreasing agent, a heat transfer media, a solvent, and/or a dielectric.

In one embodiment, an alkoxyvinyl ether has the following chemical structure:

R_{f}C(OR)═CHR_{f}′

R_{f} may be a partially fluorinated functional group or a perfluoro functional group, including, but not limited to, a partially fluorinated functional group having at least one carbon atom, a perfluoro functional group having at least one carbon atom, a partially fluorinated alkyl group, a perfluoroalkyl group, a C₁₋₁₂ partially fluorinated alkyl functional group, a C₁₋₁₂ perfluoroalkyl functional group, CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, tert-C₄F₉, or tert-C₄F₈H.

R_{f}′ may be a partially fluorinated functional group or a perfluoro functional group, including, but not limited to, a partially fluorinated functional group having at least two carbon atoms, a perfluoro functional group having at least two carbon atoms, a partially fluorinated alkyl group having at least two carbon atoms, a perfluoroalkyl group having at least two carbon atoms, a C₂₋₁₂ partially fluorinated alkyl functional group, a C₂₋₁₂ perfluoroalkyl functional group, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, tert-C₄F₉, or tert-C₄F₈H.

R is a functional group which may be, but is not limited to, a functional group having at least one carbon atom, an alkyl functional group, a C₁₋₁₂ alkyl functional group, CH₃, C₂H₅, n-C₃H₇, iso-C₃H₇, n-C₄H₉, sec-C₄H₉, iso-C₄H₉, or tert-C₄H₉.

The alkoxyvinyl ether may include any suitable combination of specific species of R, R_{f}, and R_{f}′ disclosed herein. R_{f} and R_{f}′ may be identical or distinct from one another.

In one embodiment, R_{f} is an at least partially fluorinated functional group having at least one carbon atom, R_{f}′ is an at least partially fluorinated functional group having at least two carbon atoms, and R is a functional group.

In another embodiment, R_{f} is a perfluoro functional group having at least one carbon atom, R_{f}′ is a perfluoro functional group having at least two carbon atoms, and R is a functional group having at least one carbon atom.

In yet another embodiment, R_{f} is a perfluoroalkyl functional group having at least one carbon atom, R_{f}′ is a perfluoroalkyl functional group having at least two carbon atoms, and R is an alkyl functional group.

In still another embodiment, R_{f} is a C₁₋₁₂ perfluoroalkyl functional group, R_{f}′ is a C₂₋₁₂ perfluoroalkyl functional group, and R is a C₁₋₁₂ alkyl functional group.

In another embodiment, R_{f} is selected from the group consisting of CF₃, C₂F₅, n-C₃F₇, iso-C₃F₇, n-C₄F₉, sec-C₄F₉, iso-C₄F₉, and tert-C₄F₉, R_{f}′ is selected from the group consisting of C₂F₅, n-C₃F₇, iso-C₃F₇, n-C₄F₉, sec-C₄F₉, iso-C₄F₉, and tert-C₄F₉, and R is selected from the group consisting of CH₃, C₂H₅, n-C₃H₇, iso-C₃H₇, n-C₄H₉, sec-C₄H₉, iso-C₄H₉, and tert-C₄H₉.

In yet another embodiment, R_{f} is a partially fluorinated functional group having at least one carbon atom, R_{f}′ is a partially fluorinated functional group having at least two carbon atoms, and R is a functional group having at least one carbon atom.

In still another embodiment, R_{f} is a perfluoro functional group having at least one carbon atom, R_{f}′ is a partially fluorinated functional group having at least two carbon atoms, and R is a functional group having at least one carbon atom.

In another embodiment, R_{f} is a partially fluorinated functional group having at least one carbon atom, R_{f}′ is a perfluoro functional group having at least two carbon atoms, and R is a functional group having at least one carbon atom.

In yet another embodiment, R_{f} is selected from the group consisting of CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H, R_{f}′ is selected from the group consisting of C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H, and R is selected from the group consisting of CH₃, C₂H₅, n-C₃H₇, iso-C₃H₇, n-C₄H₉, sec-C₄H₉, iso-C₄H₉, and tert-C₄H₉.

The alkoxyvinyl ether may be an E-isomer, a Z-isomer, or a mixture of E- and Z-isomers.

The alkoxyvinyl ether may have any suitable chemical structure, including, but not limited to, CF₃C(OCH₃)═CHC₂F₅, CF₃C(OC₂H₅)═CHC₂F₅, CF₃C(OC₃H₇)═CHC₂F₅, and C₂F₅C(OCH₃)═CHC₃F₇.

In one embodiment, an alkoxyvinyl ether having the structure R_{f}C(OR)═CHR_{f}′ has a reduced flammability compared to an alkoxyvinyl ether having the structure R_{f}C(OR)═CFR_{f}′. By way of example, on the basis of calculated heats of formation, CF₃C(OCH₃)═CHC₂F₅ has a heat of combustion of about 2.08 kcal/g as compared to a heat of combustion of 2.58 kcal/g for CF₃C(OCH₃)═CHCF₃. The inventive alkoxyvinyl ethers can have a heat of combustion of less than about 2.1 kcal/g and, as a result, the inventive compounds can have a low or non-flammability rating.

In one embodiment, an alkoxyvinyl ether having the structure R_{f}C(OR)═CHR_{f}′ has a reduced atmospheric lifetime compared to an alkoxyvinyl ether having the structure R_{f}C(OR)═CFR_{f}′. The inventive alkoxyvinyl ethers can have an atmospheric lifetime ranging from about 10 to 200 days and, typically, less than 100 days.

In one embodiment, an alkoxyvinyl ether having the structure R_{f}C(OR)═CHR_{f}′ has an increased polarity compared to an alkoxyvinyl ether having the structure R_{f}C(OR)═CFR_{f}′. By way of example, on the basis of calculated dipole moments, CF₃C(OCH₃)═CHC₂F₅ has a dipole moment of 2.5460 D as compared to a dipole moment of 2.3965 D for CF₃C(OCH₃)═CFC₂F₅. The inventive alkoxyvinyl ethers can have a dipole moment of about 2 to about 5 D and in some cases about 2 to about 3D.

In one embodiment, a method for preparing an alkoxyvinyl ether, includes:

R_{f}CFHCFHR_{f}′+Base/ROH→R_{f}C(OR)═CHR_{f}′

The method may include a prior step of:

R_{f}CF═CFR_{f}′+H₂→R_{f}CFHCFHR_{f}′

In another embodiment, a method for preparing an alkoxyvinyl ether, includes:

R_{f}CF═CHR_{f}′+Base/ROH→R_{f}C(OR)═CHR_{f}′

With respect to the methods for preparing the alkoxyvinyl ether: R_{f} may be a partially fluorinated functional group or a perfluoro functional group, including, but not limited to, a partially fluorinated functional group having at least one carbon atom, a perfluoro functional group having at least one carbon atom, a partially fluorinated alkyl group, a perfluoroalkyl group, a C₁-1₂ partially fluorinated alkyl functional group, a C₁₋₁₂ perfluoroalkyl functional group, CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, tert-C₄F₉, or tert-C₄F₈H; R_{f}′ may be a partially fluorinated functional group or a perfluoro functional group, including, but not limited to, a partially fluorinated functional group having at least one carbon atom, a perfluoro functional group having at least one carbon atom, a partially fluorinated alkyl group, a perfluoroalkyl group, a C₁-1₂ partially fluorinated alkyl functional group, a C₁₋₁₂ perfluoroalkyl functional group, CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, tert-C₄F₉, or tert-C₄F₈H; and R is a functional group which may be, but is not limited to, an alkyl functional group, a C₁₋₁₂ alkyl functional group, CH₃, C₂H₅, n-C₃H₇, iso-C₃H₇, n-C₄H₉, sec-C₄H₉, iso-C₄H₉, or tert-C₄H₉. The method may include any suitable combination of specific species of R, R_{f}, and R_{f}′ disclosed herein. R_{f} and R_{f}′ may be identical or distinct from one another.

In one embodiment of the invention, at least one alkyoxyl vinyl ether can interact or react with elimination of HF. In a particular embodiment, the elimination of HF can produce at least one alkyoxyl vinyl ether with an additional double bond. In one particular embodiment at least one of the following groups can eliminate HF under certain reaction conditions: iso-C₃F₆H, n-C₄F₈H, sec-C₄F₈H, iso-C₄F₈H, or tert-C₄F₈H. Without wishing to be bound by any theory or explanation, it is believed that an excess amount of base can promote the HF elimination.

The base may be any suitable base, including, but not limited to, KOH, NaOH, LiOH, Ca(OH)₂, tertiary amines, and/or alkoxides of alkali metals ROM (M=Li, Na, K, Cs). The amount of base will be at least two moles per mole of reactant. If desired, the amount of reactant can be in excess of the molar amount of base and, unreacted material (if any) can be recycled in order to increase selectivity.

In some embodiments, the reaction composition may further include any suitable solvent, including, but not limited to, alcohols, ethers (e.g., glymes, diethyl ether, methyl-t-butyl ether), tetrahydrofuran (THF), amides, and/or aromatic solvents (e.g., benzene, toluene, xylenes). The amount of solvent can range from about 0.5 to about 500 moles per mole of reactant and, in some cases, about 5 to about 100 moles of solvent.

In some embodiments, the reaction may be performed in the absence of solvent, under phase-transfer catalysis (PTC) conditions. Suitable phase transfer catalysts include, but are not limited to, tetraalkylammonium salts, phosphonium salts, and/or crown ethers. The amount of catalyst can range from about 0.1 to about 50 mole percent relative to the reactant and, in some cases, about 5 to 15 mole percent.

In some embodiments, the reaction may be performed at a temperature of at least −20 degrees Celsius, at least 0 degrees Celsius, at least 10 degrees Celsius, at least 20 degrees Celsius, at least 25 degrees Celsius, at least 30 degrees Celsius, at least 40 degrees Celsius, less than 110 degrees Celsius, less than 100 degrees Celsius, less than 90 degrees Celsius, less than 80 degrees Celsius, less than 70 degrees Celsius, less than 60 degrees Celsius, less than 50 degrees Celsius, and combinations thereof. The reaction pressure can be conducted at atmospheric pressure and, if desired, the pressure can be increased to greater than atmospheric pressure (e.g., about 1 to about 100 atmospheres).

The alkoxyvinyl ether may be useful in various applications. In an embodiment, the alkoxyvinyl ether may be used in accordance with convention equipment and methods to transfer heat. The process may include providing an article and contacting the article with a heat transfer media including the alkoxyvinyl ether. In some embodiments, the article may include electrical equipment (e.g., circuit board, computer, display, semiconductor chip, or transformer), a heat transfer surface (e.g., heat sink), or article of clothing (e.g., a body suit).

In another embodiment, the alkoxyvinyl ether may be used in a process for treating a surface. The process may include providing a surface having a treatable material deposited thereon and contacting the surface with a treatment composition including the alkoxyvinyl ether. In some embodiments, the treatment composition may substantially dissolve the treatable material. While the inventive alkoxyvinyl ethers can be used in any suitable equipment and methods, examples of such methods are disclosed by WO2012/121749 and WO2010/094019; the disclosure of which is hereby incorporated by reference.

In another embodiment, the alkoxyvinyl ether may be used in a process for forming a composition. The process includes providing a solute and contacting the solute with a solvent including the alkoxyvinyl ether. In some embodiments, the alkoxyvinyl ether may substantially dissolve the solute. Except for water, the inventive alkoxyvinyl ethers are miscible with conventional organic as well as fluorinated solvents.

In an embodiment, the alkoxyvinyl ether may be used in conventional equipment and in a process for providing electrical insulation. The process includes providing a first charged surface, providing a second charged surface, and contacting the first charged surface and the second charged surface with a dielectric composition including the alkoxyvinyl ether. In some embodiments, the dielectric composition forms a continuous pathway between the first charged surface and the second charged surface. In some embodiments, the first charged surface and the second charged surface may be substantially submerged in the dielectric composition.

The following Examples are provided to illustrate certain aspects of the invention and shall not limit the scope of the appended claims.

### EXAMPLES

### Example 1. Reaction of CF₃CFHCFHC₂F₅(I) with CH₃OH/KOH

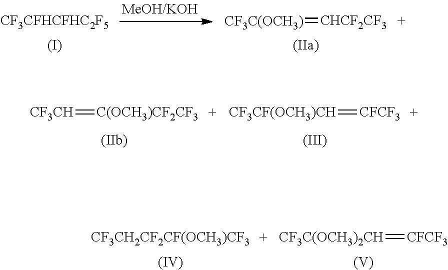

A solution of 240 g (4.27 mol) of KOH in 1400 mL of methanol was placed in a 3 L round-bottomed flask equipped with reflux condenser, thermocouple, addition funnel and magnetic stir bar. The flask was immersed in ice cooling bath. Vertrel® XF (1,1,1,2,2,3,4,5,5,5-decafluoropentane, I, 504 g, 2 mol) was added dropwise to the cooled solution, while maintaining the internal temperature below 10° C. (˜3 hours). The reaction mixture was allowed to warm to ambient temperature overnight. The next morning the reaction mixture was washed in portions with a large excess of ice water in a separatory funnel. The fluorous layers were collected, combined, and washed with a saturated solution of sodium bicarbonate (1 L×2), dried with MgSO₄ and filtered to yield 448 g of crude product. Crude products from three consecutive runs were combined (total 1085 g) and distilled at atmospheric pressure, using a 16-inch-long distillation column. Distillation data and the composition of the fractions is shown in Table 1.

TABLE 1
Distillation of Crude Reaction Mixture

| | | | Head | | | | | |
|---|---|---|---|---|---|---|---|---|
| | Weight | Pot Temp | Temp | Composition (wt %) | | | | |
| Fraction | (g) | (° C.) | (° C.) | (I) | (IIa)(IIb) | (III) | (IV) | (V) |
| 1 | 12 | 71.6-72 | 56-59.8 | 67 | 33 | — | — | — |
| 2 | 11 | 72-72.1 | 59.8-60.6 | 63 | 37 | — | — | — |
| 3 | 11 | 72.1-72.1 | 62-66.1 | 31 | 68 | — | — | — |
| 4 | 22 | 72.1-72.5 | 66.1-66.5 | 24 | 76 | — | — | — |
| 5 | 27 | 72.5-72.7 | 66.5-69.1 | 40 | 60 | — | — | — |
| 6 | 22 | 72.7-72.7 | 69.1-71.9 | 17 | 83 | — | — | — |
| 7 | 20 | 72.7-72.9 | 71.9-72 | 4.5 | 95.1 | — | — | — |
| 8 | 157 | 73.2-73.3 | 72-73.2 | 2.9 | 96.8 | — | — | — |
| 9 | 133 | 73.3-73.3 | 73.2-73.2 | — | 99.4 | — | — | — |
| 10 | 164 | 73.3-73.8 | 73.2-73.2 | — | 98.5 | 1.0 | — | — |
| 11 | 111 | 73.8-74.4 | 73.2-73.3 | — | 98.7 | 0.8 | — | — |
| 12 | 117 | 74.8-75.9 | 73.3-73.5 | — | 99.4 | 0.6 | — | — |
| 13 | 124 | 75.9-80.8 | 73.5-73.6 | — | 99.3 | 0.6 | — | — |
| 14 | 58 | 81-96 | 73.6-75 | — | 95 | 3.7 | 1 | — |
| 15 | 17 | 96-116 | 75-76.2 | — | 89 | 7.8 | 2.7 | — |
| 16 | 18 | 116-130 | 76.2-80.4 | — | 68 | 17.1 | 14.7 | — |
| POT | 44 | | | | 4 | 5 | 27 | 47 |

Combined fractions 8-13 (806 g total, yield 550%) were analyzed by NMR and shown to be a ˜99% pure mixture of compounds IIa and IIb in ratio 85:15

### Compound IIa:

¹H NMR (CDCl₃, J, Hz): 3.98 (3H, s), 5.74 (1H, q, 8.4), ppm

¹⁹F NMR (CDCl₃, J, Hz): −55.34 (3F, d, 8.1), −83.23 (3F, t, 1.5), −119.34 (2F, s) ppm

### Compound IIb:

¹H NMR (CDCl₃, δ, J, Hz): δ 3.92 (3H, s), 5.57 (1H, t, 13.3) ppm

¹⁹F NMR (CDCl₃, J, Hz): −69.42 (3F, s,), −85.78 (3F, t, 2.5), −110.70 (2F, dt, 13.8, 2.5) ppm

### Compound III:

¹H NMR (CDCl₃, J, Hz): 3.58 (3H, s), 5.62 (1H, dd, 28.1, 15.6) ppm

¹⁹F (CDCl₃, J, Hz): −60.66 (3F, m, 9.9, 4.5), −80.36 (3F, d, 8.8), −118.90 (1F, tm, 28.1), −127.24 (1F, dd, 28.1, 15.3) ppm

### Compound IV:

¹H NMR (CDCl₃, J, Hz): 2.84 (2H, m), 3.62 (3H, q, 1.2) ppm

¹⁹F (CDCl₃, J, Hz): −73.41 (3F, d, 9.9), −83.86 (3F, t, 4.5), −124.71 (2F, A:B q, 286.0), −130.45 (1F, m, 9.1) ppm

### Compound V:

¹H NMR (CDCl₃, J, Hz): 3.41 (6H, s), 5.48 (1H, d, 33.2) ppm

¹⁹F (CDCl₃, J, Hz): −73.24 (3F, d, 9.3), −78.23 (3F, d, 6.8), −120.87 (1F, dm, J_{d}=33.2) ppm

### Example 2. Reaction of CF₃CFHCFHC₂F₅ with CF₃CH₂OH/KOH

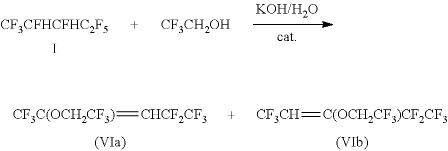

A 500 mL 3-neck round bottomed flask was equipped with an addition funnel, thermocouple, and reflux condenser. Under a nitrogen blanket, 200 mL of 45 wt % KOH/water solution was added, along with 2 g of catalyst—tetrabutylammonium bromide (TBAB, phase transfer catalyst). This mixture was cooled in an ice bath down to −5° C. and 50 g (˜36 mL, 0.5 mol) of trifluoroethanol was added dropwise over 30 mins period at 10° C. Addition of trifluoroethanol was followed by slow addition of 125 g (0.5 mol) of I at 5-10° C. (˜30 min). The reaction mixture was slowly brought to ambient temperature (˜4 hrs). After stirring at ambient temperature for an additional ˜3 hrs, the reaction mixture was poured into 500 mL of water, fluorous layer separated, washed with water, dried over MgSO₄, filtered and distilled using a Vigreux column. 87 g (yield 56%) of fraction b.p. 95-98° C. was isolated. The fraction contained isomers VIa and VIb in a ratio of 72:25, respectively and about 3% of other isomeric products.

### Compound VIa:

¹H NMR (CDCl₃, J, Hz): 4.39 (2H, q, 7.7) 5.98 (1H, q, 7.6,), ppm

¹⁹F NMR (CDCl₃, J, Hz): δ −57.32 (3F, dq, 7.5, 1.7), −75.19 (3F, t, 7.8), −83.16 (3F, d, 0.7), −118.83 (2F, s) ppm

### Compound VIb:

¹H NMR (CDCl₃, J, Hz): δ 5.80 (1H, t, 12.9), 4.36 (2H, q, 7.9) ppm

¹⁹F NMR (CDCl₃, J, Hz): −69.20 (3F, s), −75.1 (3F, t, 7.6), −85.54 (3F, t, 2.3), −112.23 (2, F dm, 12.9, 2.1) ppm

### Example 3. Reaction of CF₃CFHCFHC₂F₅ with CH₃CH₂OH/KOH

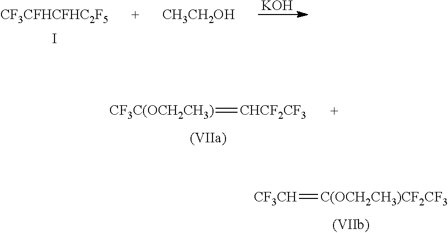

Using the procedure described above in Example 1, 126 g (0.5 mol) of I, was added to a solution of 70 g (1.07 mol) of KOH in 400 ml of ethanol. The resulting crude product isolated after washing reaction mixture with water, drying, filtration and distillation yielded 94.4 g (yield 73%) of material with b.p. 85-89° C., which was found to be a mixture of VIIa and VIIb in a ratio 75:25, containing 3% isomeric material (NMR and GC/MS).

### Compound VIIa:

¹H NMR (CDCl₃, J, Hz): 1.36 (3H, t, 7.4), 4.19 (2H, q, 7.4), 5.74 (1H, q, 8.0) ppm

¹⁹F NMR (CDCl₃, J, Hz): −56.34 (3F, dt, 8.1, 2.2), −83.18 (3F, t, 1.9), −119.23 (2F, s) ppm

### Compound VIIb:

¹H NMR (CDCl₃, J, Hz): 1.35 (3H, t, 7.3), 4.14 (2H, q, 7.3), 5.74 (1H, t, 12.8) ppm

¹⁹F NMR (CDCl₃, J, Hz): −69.58 (3F, s), −85.81 (3F, t, 2.5), −111.27 (2F, dm, J_{d}=13.8) ppm

### Example 4. Reaction of CF₃CFHCFHC₂F₅ with (CH₃)₂CHOH/KOH

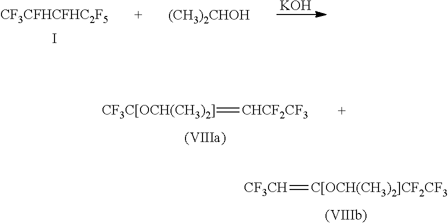

Following the procedure described above in Example 1. Compound I (126 g, 0.5 mol) was added to a solution of 70 g (1.07 mol) of KOH in 400 ml of iso-propanol. The isolated crude product was distilled to yield 79 g (yield 61%) of a fraction with b.p. 98-99° C., containing a mixture of VIIa and VIIb in a ratio of ˜70:30, containing 3% of other isomeric material (NMR, GC/MS).

### Compound VIIIa:

¹H NMR (CDCl₃, J, Hz): 1.31 (6H, d, 6.0), 4.63 (1H, quint., 6.0), 5.76 (1H, q, 8.0,) ppm

¹⁹F NMR ((CDCl₃, J, Hz): −57.54 (3F, dt, 8.0, 2.1), −83.00 (3F, t, 1.9), −117.99 (2F, s) ppm

### Compound VIIIb:

¹H NMR (CDCl₃, J, Hz): 1.31 (6H, d, 6.0), 4.56 (1H, quint., 6.0), 5.56 (1H, t, 13.0) ppm

¹⁹F NMR (CDCl₃, J, Hz): −68.38 (3F, s), −85.57 (3F, t, 2.5), −111.55 (2F, dq, 13.0, 2.2) ppm

### Example 5. Reaction of C₃F₇CH═CFC₂F₅/C₃F₇CF═CHC₂F₅ with MeOH/KOH

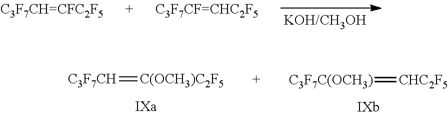

Following the procedure described above in Example 1. The olefin (mixture of C₃F₇CH═CFC₂F₅ and C₃F₇CF═CHC₂F₅ in ratio ˜1:1) was added to a solution of KOH (20 g, 0.36 mol) in 300 ml of MeOH. After work up 105 g of crude product was isolated and distilled to yield 61.5 g (yield 46%), of a fraction with b.p. 107-110° C., containing a mixture of IXa, IXb, and isomeric material in ratio 45:40:15 (GC/MS, NMR)

### Major Isomer (Content—45% in a Mixture):

¹H NMR (CDCl₃, J, Hz): 3.96 (3H, t, 1.2), 5.66 (1H, t, 14.5) ppm

¹⁹F NMR (CDCl₃, J, Hz): −81.14 (3F, t, 9.5), −85.02 (3F, t, 1.9), −109.06 (2F, dm, J_{d}=14.5), −116.39 (2F, q, 9.5), −127.05 (2F, s) ppm

### Minor Isomer (Content—40% in a Mixture):

¹H NMR (CDCl₃, J, Hz): 3.92 (3H, t, 1.2), 5.73 (1H, t, 14.0) ppm

¹⁹F NMR (CDCl₃, J, Hz): δ −80.78 (3F, t, 9.2), −83.55 (3F, m), −107.16 (2F, quint., 11.0), −118.55 (2F, s), −128.09 (2F, s) ppm

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An alkoxyvinyl ether having the following chemical structure:
R_{f}C(OR)═CHR_{f}′
wherein:
R_{f} is an at least partially fluorinated functional group having at least one carbon atom;
R_{f}′ is selected from the group consisting of C₂F₅, n-C₃F₇, iso-C₃F₇, n-C₄F₉, sec-C₄F₉, iso-C₄F₉, tert-C₄F₉, and a partially fluorinated functional group having at least two carbon atoms; and
R is a functional group having at least one carbon atom.

2. The alkoxyvinyl ether of claim 1, wherein:
R_{f} is a perfluoro functional group having at least one carbon atom; and
R_{f}′ is selected from the group consisting of C₂F₅, n-C₃F₇, iso-C₃F₇, n-C₄F₉, sec-C₄F₉, iso-C₄F₉, and tert-C₄F₉.

3. The alkoxyvinyl ether of claim 2, wherein:
R_{f} is a perfluoroalkyl functional group having at least one carbon atom; and
R is an alkyl functional group.

4. The alkoxyvinyl ether of claim 3, wherein:
R_{f} is a C₁₋₁₂ perfluoroalkyl functional group; and
R is a C₁₋₁₂ alkyl functional group.

5. The alkoxyvinyl ether of claim 4, wherein:
R_{f} is selected from the group consisting of CF₃, C₂F₅, n-C₃F₇, iso-C₃F₇, n-C₄F₉, sec-C₄F₉, iso-C₄F₉, and tert-C₄F₉; and
R is selected from the group consisting of CH₃, C₂H₅, n-C₃H₇, iso-C₃H₇, n-C₄H₉, sec-C₄H₉, iso-C₄H₉, and tert-C₄H₉.

6. The alkoxyvinyl ether of claim 1, wherein:
R_{f} is a partially fluorinated functional group having at least one carbon atom;
R_{f}′ is a partially fluorinated functional group having at least two carbon atoms; and
R is an alkyl functional group.

7. The alkoxyvinyl ether of claim 1, wherein:
R_{f} is a perfluoro functional group having at least one carbon atom; and
R_{f}′ is a partially fluorinated functional group having at least two carbon atoms.

8. The alkoxyvinyl ether of claim 1, wherein:
R_{f} is a partially fluorinated functional group having at least one carbon atom; and
R_{f}′ is selected from the group consisting of C₂F₅, n-C₃F₇, iso-C₃F₇, n-C₄F₉, sec-C₄F₉, iso-C₄F₉, tert-C₄F₉.

9. The alkoxyvinyl ether of claim 1, wherein:
R_{f} is selected from the group consisting of CF₃, CF₂H, C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H;
R_{f}′ is selected from the group consisting of C₂F₅, C₂F₄H, n-C₃F₇, n-C₃F₆H, iso-C₃F₇, iso-C₃F₆H, n-C₄F₉, n-C₄F₈H, sec-C₄F₉, sec-C₄F₈H, iso-C₄F₉, iso-C₄F₈H, and tert-C₄F₉, tert-C₄F₈H; and
R is selected from the group consisting of CH₃, C₂H₅, n-C₃H₇, iso-C₃H₇, n-C₄H₉, sec-C₄H₉, iso-C₄H₉, and tert-C₄H₉.

10. The alkoxyvinyl ether of claim 1, wherein R_{f} and R_{f}′ are the same.

11. The alkoxyvinyl ether of claim 1, wherein R_{f} and R_{f}′ are distinct.

12. The alkoxyvinyl ether of claim 1, wherein the alkoxyvinyl ether is an E-isomer.

13. The alkoxyvinyl ether of claim 1, wherein the alkoxyvinyl ether is a Z-isomer.

14. The alkoxyvinyl ether of claim 1, wherein the alkoxyvinyl ether is a mixture of E- and Z-isomers.

15. The alkoxyvinyl ether of claim 1, wherein the chemical structure is CF₃C(OC₂H₅)═CHC₂F₅.

16. The alkoxyvinyl ether of claim 1, wherein the chemical structure is CF₃C(OC₃H₇)═CHC₂F₅.

17. The alkoxyvinyl ether of claim 1, wherein the chemical structure is C₂F₅C(OCH₃)═CHC₃F₇.

18. The alkoxyvinyl ether of claim 1, wherein the chemical structure is CF₃C(OCH₂CH₃)═CHC₂F₅.

19. The alkoxyvinyl ether of claim 1, wherein the chemical structure is CF₃C(OCH(CH₃)₂)═CHC₂F₅.

20. An alkoxyvinyl composition comprising:
the alkoxyvinyl ether of claim 1;
a solvent including at least one of alcohols, ethers, tetrahydrofuran (THF), amides, or aromatic solvents; and
a base including at least one of KOH, NaOH, LiOH, Ca(OH)₂, tertiary amines, or alkoxides of alkali metals ROM (M=Li, Na, K, Cs).

21. A process for transferring heat, comprising:
providing an article; and
contacting the article with a heat transfer media;
wherein the heat transfer media comprises the alkoxyvinyl ether of claim 1.

22. The process of claim 21, wherein the boiling point of the alkoxyvinyl ether of claim 1 is between 72° C. and 81° C.

23. A process for treating a surface, comprising:
providing a surface; and
contacting the surface with a treatment composition;
wherein the surface includes a treatable material deposited thereon; and
wherein the treatment composition comprises the alkoxyvinyl ether of claim 1.

24. The process of claim 23, wherein the treatment composition substantially dissolves the treatable material.

25. A process for forming a composition comprising:
providing a solute; and
contacting the solute with a solvent;
wherein the solvent comprises the alkoxyvinyl ether of claim 1.

26. A process for providing electrical insulation, comprising:
providing a first charged surface;
providing a second charged surface; and
contacting the first charged surface and the second charged surface with a dielectric composition;
wherein the dielectric composition comprises the alkoxyvinyl ether of claim 1.

27. The process of claim 26, wherein the dielectric composition forms a continuous pathway between the first charged surface and the second charged surface.
